(19) United States
(12) Patent Application Publication
(10) Pub. No.: US 2022/0283181 A1
(43) Pub. Date: Sep. 8, 2022

(54) METHOD FOR PREDICTING PROGNOSIS FOR CANCER PATIENT, METHOD FOR PREDICTING EFFECTIVENESS OF ANTICANCER THERAPY, AND METHOD FOR SELECTING APPROPRIATE THERAPY FOR CANCER PATIENT

(71) Applicants: MEIJI CO., LTD., Tokyo (JP); THE NISSHIN OILLIO GROUP, LTD., Tokyo (JP); OSAKA UNIVERSITY, Osaka (JP)

(72) Inventors: Keisuke HAGIHARA, Osaka (JP); Satoshi OSAGA, Osaka (JP); Noriyuki IKEDA, Kanagawa (JP); Satoshi TAKASUGI, Tokyo (JP); Kinya ASHIDA, Tokyo (JP); Kentaro NAKAMURA, Tokyo (JP)

(73) Assignees: MEIJI CO., LTD., Tokyo (JP); THE NISSHIN OILLIO GROUP, LTD., Tokyo (JP); OSAKA UNIVERSITY, Osaka (JP)

(21) Appl. No.: 17/624,948

(22) PCT Filed: Jul. 7, 2020
(86) PCT No.: PCT/JP2020/026589

§ 371 Date: Jan. 5, 2022

(30) Foreign Application Priority Data
Jul. 8, 2019 (JP) 2019-127042

(51) Int. Cl. G01N 33/68; G01N 33/66

(52) U.S. Cl. CPC G01N 33/6893; G01N 33/66; G01N 2800/52

(57) ABSTRACT

An object of the present invention is to provide a method for predicting prognosis for a cancer patient. The present invention provides a method for predicting prognosis for a cancer patient, wherein at least one index selected from the group consisting of an index of nutritional state, an index of sugar metabolism state, and an index of inflammatory state in the cancer patient is used as an index of prognosis. The index of nutritional state is preferably a concentration of albumin in the blood, the index of sugar metabolism state is preferably a concentration of glucose in the blood, and the index of inflammatory state is preferably a concentration of CRP in the blood.

### CROSS-REFERENCE TO RELATED APPLICATION

The present application enjoys the benefit of priority from the prior Japanese Patent Application No. 2019-127042 filed on. Jul. 8, 2019, the entire disclosure of which is incorporated herein by reference.

### TECHNICAL FIELD

The present invention relates to a method for predicting prognosis for a cancer patient. The present invention also relates to a method for predicting effectiveness of an anticancer therapy in a cancer patient and a method for selecting an appropriate therapy for a cancer patient. The present invention further relates to a method for predicting prognosis for a cancer patient in a ketogenic diet therapy. The present invention also relates to a method for predicting effectiveness of a ketogenic diet therapy in a cancer patient and a method for selecting an appropriate therapy for a cancer patient in a ketogenic diet therapy.

### BACKGROUND ART

Recently, along with Westernization of eating habits, cancers often found in Western countries, such as colorectal cancer, breast cancer, lung cancer and prostate cancer, have increased instead of gastric cancer. In the case of carcinomas, such as gastric cancer and colorectal cancer, which are resectable upon early detection, patients' life prognoses are being improved. However, many of refractory cancers including pancreatic cancer and osteosarcoma are difficult to early detect, and clinical countermeasures exist as great problems. Existing cancer treatment mainly involves surgical resection, a chemotherapy and a radiation therapy, and the chemotherapy cannot be said to provide a sufficient therapeutic effect on lung cancer and pancreatic cancer. Accordingly, there exists a further demand for development of effective therapies for cancer patients, and, amongst others, a novel therapy for refractory cancer patients is strongly demanded.

Ketone body is a generic term for acetoacetic acid, β-hydroxybutyric acid and acetone, and is synthesized in the liver by β-oxidation of fatty acids in the living body. In humans, when glucose is insufficiently supplied due to fasting state or long-term exercise, fat is decomposed to produce ketone bodies from fatty acids, and the ketone bodies are used as an energy source (Non-Patent Document 1). As a meal devised to produce many ketone bodies in the body, a ketogenic diet, which is a high-lipid low-saccharide diet, has been traditionally known. Upon intake of a ketogenic diet, the concentration of ketone bodies in the blood rises. It has been reported in the past that ketogenic diet therapies are effective for treatment of cancer (Patent Document 1), but there is no simple index from which the effect of a ketogenic diet therapy in cancer patients can be predicted.

### REFERENCE LIST

### Patent Documents

Patent Document 1: WO 2017/038101

### Non-Patent Documents

Non-Patent Document 1: Vidali S, et al., Int J Biochem Cell Biol. 63, 55-59 (2015)

### SUMMARY OF THE INVENTION

An object of the present invention is to provide a method for predicting prognosis for a cancer patient and a method for predicting effectiveness of an anticancer therapy in a cancer patient. Another object of the present invention is to provide a method for selecting an appropriate therapy for a cancer patient. Still another object of the present invention is to provide a method for predicting prognosis for a cancer patient on whom a ketogenic diet therapy is performed and a method for predicting effectiveness of the ketogenic diet therapy in the patient. Even still another object of the present invention is to provide a method for selecting an appropriate therapy for a cancer patient in a ketogenic diet therapy.

The present inventors have now investigated indexes regarding the nutritional state, sugar metabolism state, and inflammatory state in a cancer patient, and found that these indexes can be used to predict prognosis for the cancer patient, to predict the effect of an anticancer therapy in the cancer patient, and further to select an appropriate therapy for the cancer patient. The present inventors have also found that a concentration of albumin in the blood, a blood sugar level, and a concentration of CRP in the blood can be used as indexes in a terminal cancer patient on whom a saccharide restricted high fat diet therapy has been performed to predict prognosis for the cancer patient, to predict the effect of a ketogenic diet therapy in the cancer patient, and further to select a therapy in the cancer patient. The present invention is based on these findings.

The present invention provides the following inventions.

[1] A method for predicting prognosis for a cancer patient, wherein at least one index selected from the group consisting of an index of nutritional state, an index of sugar metabolism state, and an index of inflammatory state in the cancer patient is used as an index of prognosis.

[2] A method for predicting effectiveness of an anticancer therapy in a cancer patient, wherein at least one index selected from the group consisting of an index of nutritional state, an index of sugar metabolism state, and an index of inflammatory state in the cancer patient is used as an index of effectiveness of an anticancer therapy in the cancer patient.

[3] A method for selecting an appropriate therapy for a cancer patient, wherein at least one index selected from the group consisting of an index of nutritional state, an index of sugar metabolism state, and an index of inflammatory state in the cancer patient is used as an index for selection of an appropriate therapy for the cancer patient.

[4] A method for treating a cancer patient, comprising the steps of: carrying out the method according to [3] to select an appropriate therapy for the cancer patient; and performing the selected therapy on the patient.

[5] The method according to any one of [1] to [4], wherein a combination of the index of nutritional state, the index of sugar metabolism state, and the index of inflammatory state is used as the index.

[6] The method according to any one of [1] to [5], wherein the index of nutritional state is a concentration of albumin in the blood.

[7] The method according to any one of [1] to [6], wherein the index of sugar metabolism state is a concentration of glucose in the blood.

[8] The method according to any one of [1] to [7], wherein the index of inflammatory state is a concentration of C-reactive protein (CRP) in the blood.

[9] The method according to any one of [1] to [8], wherein the cancer patient is a refractory cancer patient having a performance status of 2 or less.

[10] The method according to any one of [1] and [5] to [9], wherein the cancer patient is a patient on whom a ketogenic diet therapy has been performed.

[11] The method according to any one of [2] and [5] to [9], wherein the cancer patient is a patient on whom a ketogenic diet therapy has been performed.

[12] The method according to any one of [3] and [5] to [9], wherein the cancer patient is a patient on whom a ketogenic diet therapy has been performed.

[13] The method according to any one of [4] to [9], wherein the cancer patient is a patient on whom a ketogenic diet therapy has been performed.

[14] The method according to any one of [10] to [13], wherein the ketogenic diet therapy is performed according to the following procedures:

(a) for the first week, meals are provided to the subject at a daily calorie of about 1500 kcal and at a ratio of about 140 g of lipid:about 60 g of protein:about 10 g of saccharide (carbohydrate other than dietary fibers), based on a real body weight of 50 kg;

(b) from the second week to the third month, meals are provided to the subject at a daily intake amount of saccharide of about 20 g or less, at a daily calorie of about 1400 to about 1600 kcal, and at a ratio of about 120 to about 140 g of lipid:about 70 g of protein:about 20 g of saccharide; and

(c) after the third month, meals are provided to the subject at a daily intake amount of saccharide of about 30 g or less, at a single intake amount of saccharide of about 10 g/intake, and in accordance with (b) with respect to others.

Hereinafter, the methods for predicting according to [1] and [10] are sometimes referred to simply as “the prognosis prediction methods of the present invention.” Hereinafter, the methods for predicting according to [2] and [11] are sometimes referred to simply as “the methods for predicting effective of an anticancer therapy of the present invention.” Hereinafter, the methods for selecting according to [3] and [12] are sometimes referred to simply as “the selection methods of the present invention.” Hereinafter, the methods for treating according to [4] and [13] are sometimes referred to simply as “the treatment methods of the present invention.”

According to the present invention, there are provided a method for predicting prognosis for a cancer patient, a method for predicting effectiveness of an anticancer therapy in a cancer patient, and a method for selecting an appropriate therapy for a cancer patient, wherein at least one of an index of nutritional state, an index of glucose metabolism state, and an index of inflammatory state is used as an index. Also, according to the present invention, there are provided a method for predicting prognosis for a cancer patient in a ketogenic diet therapy, a method for predicting effectiveness of a ketogenic diet therapy in a cancer patient, and a method for selecting an appropriate therapy for a cancer patient in a ketogenic diet therapy, wherein at least one of an index of nutritional state, an index of glucose metabolism state, and an index of inflammatory state is used as an index.

### BRIEF DESCRIPTION OF THE DRAWINGS

FIG. 1 is a diagram showing a survival rate curve by the Kaplan-Meier method using, as an index, a combination of a concentration of albumin in the blood, a blood sugar level, and a concentration of CRP in the blood.

### DETAILED DESCRIPTION OF THE INVENTION

### Definition

### <Ketogenic Diet Therapy>

In the present invention, the “ketogenic diet” means a “saccharide restricted high fat diet,” and the “ketogenic diet therapy” means a therapy based on feeding a ketogenic diet to a subject.

Here, the “high fat diet” refers to a diet which allows intake of fat corresponding to about 30% or more of energy based on the total energy intake. This numerical value is based on the fact that 30% or more of the total energy intake is normally derived from lipid, according to the National Health and Nutrition Survey 2005 and 2006. The “high fat diet” can be defined by the proportion of energy corresponding to a fat intake amount based on the total energy intake. The lower limit value of the proportion can be set to about 50%, about 55%, about 60%, about 65% or about 70%, and the upper limit value of the proportion can be set to about 95%, about 90%, about 85% or about 80%. The lower limit value and upper limit value can be combined arbitrarily to define a numerical range. The “high fat diet” can allow intake of fat corresponding to about 50% to about 95%, about 60% to about 90%, about 65% to about 85% or about 70% to about 80% of energy based on the total energy intake. It should be noted that the energy ratio is calculated on the premise that 1 g of fat produces 9 kcal.

Also, the “high fat diet” refers to a diet which allows intake of fat in an amount of about 80 g or more per day based on a real body weight of 50 kg. The “high fat diet” can be defined by the fat intake amount per day based on a real body weight of 50 kg. The lower limit value of the fat intake amount can be set to about 80 g, about 85 g, about 90 g, about 95 g, about 100 g, about 105 g, about 110 g, about 115 g or about 120 g, and the upper limit value of the fat intake amount can be set to about 180 g, about 175 g, about 170 g, about 165 g, about 160 g, about 155 g, about 150 g, about 145 g or about 140 g. The lower limit value and upper limit value can be combined arbitrarily to define a numerical range. The “high fat diet” can allow intake of fat in an amount of about 80 g to about 180 g per day, about 90 g to about 170 g per day, about 100 g to about 160 g per day, about 110 g to about 150 g per day, or about 120 g to about 140 g per day based on a real body weight of 50 kg.

Also, the “saccharide restricted” refers to feeding about 100 g or less of saccharide per day based on a real body weight of 50 kg. This numerical value is calculated based on the description that “Supposing that the basal metabolic rate is 1,500 kcal/day, the energy consumption of the brain is 300 kcal/day which corresponds to 75 g/day of glucose. Since tissues other than the brain also utilize glucose as an energy source as described above, the necessary amount of glucose is estimated to be at least 100 g/day, i.e., the minimum necessary amount of digestible carbohydrate is estimate to be approximately 100 g/day” in the nutrition report in 2010 by the Ministry of Health, Labour and Welfare. It is understood that this numerical value can vary. The “saccharide restricted diet” can be defined by the saccharide intake amount per day based on a real body weight of 50 kg. The lower limit value of the saccharide intake amount can be set to about 5 g, about 10 g, about 15 g, about 20 g, about 25 g or about 60 g, and the upper limit value of the saccharide intake amount can be set to about 70 g, about 35 g, about 30 g, about 25 g, about 20 g or about 15 g. The lower limit value and upper limit value can be combined arbitrarily to define a numerical range. The “saccharide restricted diet” can allow intake of saccharide in an amount of about 60 g to about 70 g per day, about 5 g to about 15 g per day, about 15 g to about 25 g per day, or about 25 g to about 35 g per day based on a real body weight of 50 kg.

In a preferred embodiment of the ketogenic diet in the present invention, the intake amount of saccharide in the introduction period may be further restricted. For example, the intake amount of saccharide may be restricted to about 20 g/day or less or about 10 g/day or less. The intake amount of saccharide in the introduction period is further limited, thereby making it possible rapid induction of blood ketone bodies (acetoacetic acid and β-hydroxybutyric acid). However, the contents of meals at the early stage of introduction are different from conventional eating habits, and thus are difficult to continue. Gradual relaxation of the restriction on the saccharide intake amount makes it possible to continue the ketogenic diet, and a therapeutic effect is also observed. Accordingly, the restricted amount of saccharide (intake amount of carbohydrate) is not limited, for example, to about 10 g/day->about 20 g/day->about 30 g/day, so long as the restriction on saccharide is characterized by being gradually relaxed from a strict restrict in the initial amount (for example, about 10 g/day or less). Thus, the amount of saccharide to be initially introduced can be started at about 5 g/day to about 15 g/day or thereabouts (±about 5 g/day) in some cases. At the second stage, the amount of saccharide can be maintained at about 15 g/day to about 25 g/day or thereabouts (±about 5 g/day). At the last maintenance stage, the amount of saccharide can be maintained at about 25 g/day to about 35 g/day or thereabouts (±about 10 g/day).

The ketogenic diet in the present invention can be defined based on the ketogenic ratio (lipid/(protein+saccharide)) (by mass). Here, the ketogenic diet in the present invention includes a diet having a ketogenic ratio of about 1 or more (preferably about 2 or more, more preferably about 2.5 or more), and the upper limit value of the ketogenic ratio can be, for example, about 4 or about 3.5. Also, the ketogenic ratio may be set, for example, to about 1 to about 2, and can be about 2 at the time of introduction. Any amounts of protein and saccharide can be employed so long as the ketogenic ratio satisfies this definition. The amounts of protein and saccharide are preferably about 30 g or less per day, more preferably about 20 g or less per day, further preferably about 10 g or less per day, or may be a combination thereof depending on the period. The single intake amount may be within any range so long as it falls within the daily intake amount range, and can preferably be set to about 10 g or less.

Preferred embodiments of the ketogenic diet in the present invention include Ketonformula (817-B) (manufactured by Meiji Co., Ltd.) and a composition comprising components equivalent to those of the ketogenic formula as well as their modified products (for example, a product obtained by further reducing saccharide and/or protein in Ketonformula (817-B) (for example, a product in which the amounts of the respective components in Ketonformula (817-B) are independently changed by ±about 5%, ±about 10%, ±about 15%, ±about 20% or ±about 25%)).

In the ketogenic diet in the present invention, medium chain fatty acid oils may be used in combination. As used herein, the “medium chain fatty acid oil” means oil in which constituent fatty acids of oil and fat have a medium chain length, which is also called MCT (Medium Chain Triglyceride) or medium chain fatty acid triglyceride, and refers to oil composed of fatty acids typically with 6 to 12 carbon atoms, preferably with 8 to 12 carbon atoms, or oil composed of fatty acids with 8 to 11 carbon atoms or fatty acids with 8 to 10 carbon atoms.

Since medium chain fatty acid oils exist in oils and fats contained in plant bodies including Palmae plants such as coconut and palm fruit and dairy products such as cow's milk, medium chain fatty acid oils extracted (including rough extraction) or purified (including rough purification) from these oils and fats (preferably, vegetable oils and fats such as palm kernel oil) can be used as they are or as raw materials. Alternatively, products and commercial products by chemical synthesis methods may each be used as medium chain fatty acid oils. As the medium chain fatty acid oil, Nisshin MCT oil, Nisshin MCT powder (both manufactured by The Nisshin OilliO Group, Ltd.) and extra virgin coconut oil (manufactured by The Nisshin OilliO Group, Ltd.) can be used.

In the present invention, the ketogenic diet therapy can be performed by continuously feeding the ketogenic diet as described above to the subject for a predetermined period (for example, 3 months). In the ketogenic diet therapy in the present invention, necessary trace elements and vitamins can be fed to the subject through supplements or the like.

In the present invention, the ketogenic diet therapy can be performed, for example, in the following manner.

(1) For the first week, the goal is: a calorie of about 30 kcal/kg body weight, no restriction on lipid or protein, and about 10 g or less of saccharide (carbohydrate other than dietary fibers), based on a real body weight. Specifically, based on a real body weight of 50 kg at the early stage of introduction, the goal is: a daily caloric intake of about 1500 kcal and a ratio of about 140 g of lipid:about 60 g of protein:about 10 g of saccharide, and a ketogenic ratio (lipid/(protein+saccharide)) of 2. Other nutrients can be fed without restriction. Necessary trace elements and/or vitamins are appropriately fed using a supplement or the like. The period can be appropriately extended/shortened, and may be set to several days to several weeks.

(2) For the second week to the third month, the saccharide amount and the intake amount of medium chain fatty acids by a ketogenic formula and an MCT oil are adjusted with reference to the blood ketone body values. For example, guidance is given so that acetoacetic acid is not 500 μmol/L, or more and β-hydroxybutyric acid is not 1000 μmol/L or less, and, if possible, the target values are set to 1000 μmol/L or more for acetoacetic acid and 2000 μmol/L or more for β-hydroxybutyric acid. The goal is: a daily intake amount of saccharide of about 20 g or less, a daily calorie of about 1400 to about 1600 kcal, a ratio of about 120 to about 140 g of lipid:about 70 g of protein:about 20 g of saccharide, and a ketogenic ratio of about 1 to about 2. At the time of calorie supply, an MCT oil and a ketogenic formula can preferably be used. The period may appropriately be extended/shortened, which may be slightly longer or shorter than two weeks, and the third month may also be slightly shifted ahead or behind (about one, two or several weeks of shift is acceptable).

(3) After the third month, the daily intake amount of carbohydrate is set to about 30 g or less when the single intake amount thereof is 10 g/day, and others are in accordance with the above (2). Since this period comes after (2), this period itself would be shifted when the third month is shifted.

### <Cancer>

The “cancer” is used herein in the meaning including tumors developed due to mutation of normal cells. The cancer can arise from any organ or tissue throughout the body. As used herein, the “cancer” includes cancers such as lung cancer, ovarian cancer, bladder cancer, labial gland-like cystic cancer, renal cancer, urinary tract epithelial cancer, colorectal cancer, prostate cancer, glioblastoma multiforme, pancreatic cancer, breast cancer, melanoma, liver cancer, gastric cancer, and esophageal cancer, and the cancer patient means a person suffering from cancer.

As used herein, the “anticancer therapy” means a therapy for cancer. Examples of the “anticancer therapy” include surgical therapies such as resection/extraction, chemotherapies, radiation therapies, cancer immunotherapies such as CAR-T cell therapies, and diet therapies such as ketogenic diet therapies, and combinations of some or all of them are also included therein.

The cancer patient who is a target for the anticancer therapy (particularly, ketogenic diet therapy) in the present invention can be a refractory cancer patient. Refractory cancer includes cancers of types and stages conventionally regarded as being difficult to treat, such as stage IV terminal cancer, hardly resectable cancer, cancer difficult to early detect and metastatic cancer. The refractory cancer patient is, for example, a refractory cancer patient having a performance status of 2 or less.

### <<Method for Predicting Prognosis>>

In the prognosis prediction method of the present invention, at least one index selected from the group consisting of an index of nutritional state, an index of sugar metabolism state, and an index of inflammatory state is used as an index of prognosis.

As used herein, the “index of nutritional state” means an index which correlates with the nutritional state, and refers to an index which can be measured or calculated by a blood test, a urine test, physical measurement, physical fitness measurement, motor function measurement, or the like. The index of nutritional state is not particularly limited as long as it is an index which correlates with the nutritional state. Examples of the index of nutritional state include substances which are contained in biological samples such as blood and urine obtained from the subject and correlate with the nutritional state, or concentrations thereof in the biological samples, and more specific examples thereof include blood concentrations and urine concentrations of albumin, prealbumin (transthyretin), transferrin, retinol-binding protein (RPB), and various amino acids and derivatives thereof (3-methylhistidine, BCAA, leucine, isoleucine, valine, etc.). Further, the index which correlates with the nutritional state may be an index which is measured or calculated by physical measurement, physical fitness measurement, motor function measurement or the like of the subject, and correlates with the nutritional state. Examples of the index include lean body mass, muscle mass, body mass index (BMI), lean body mass rate, muscle strength (grip strength, knee extension muscle strength, etc.), skeletal muscle index (SMI), phase angle (PA), finger ring test, normal walking speed, maximum walking speed, timed up and go (TUG) test, frailty index, and sarcopenia index. Further, as the index of nutritional state, any one of the indexes which correlate with the nutritional state may be used, or a plurality of the indexes which correlate with the nutritional state may be used in combination. For example, not only the concentration of albumin in the blood but also the lean body mass, the muscle strength and the like can be combined as the index of nutritional state.

As used herein, the “index of sugar metabolism state” means an index which correlates with the sugar metabolism state, and refers to an index which can be measured or calculated by a blood test, a urine test, or the like. The index of sugar metabolism state is not particularly limited as long as it is an index which correlates with the sugar metabolism state. Examples of the index of sugar metabolism state include substances which are contained in biological samples such as blood and urine obtained from the subject and correlate with the sugar metabolism state, or concentrations thereof in the biological samples, and more specific examples thereof include fasting blood sugar level, casual blood sugar level, and blood concentrations and urine concentrations of insulin, glucagon, C peptide, incretin, glucose-dependent insulinotropic polypeptide (GIP: Gastric Inhibitory Polypeptide), GLP-1, adipocytokine, leptin, adiponectin, AGEs, HbA1c, glycoalbumin, 1,5-anhydroglucitol, fructosamine and the like. In addition, the index which correlates with the sugar metabolism state may be an index which correlates with the insulin resistance and insulin secretory capacity of the subject. Examples of the index include AUC, Cmax and Tmax of glucose or insulin, blood sugar level/insulin after 120 minutes, insulin index, HOMA-IR, insulin resistance/sensitivity index, sugar uptake capacity index, and HOMA-β (insulin secretory capacity index) by a sugar tolerance test. Further, as the index of sugar metabolism state, any one of the indexes which correlate with the sugar metabolism state may be used, or a plurality of the indexes which correlate with the sugar metabolism state may be used in combination. For example, not only the fasting blood sugar level but also the blood insulin concentration, the HbAlc concentration and the like can be combined as the index of sugar metabolism state.

As used herein, the “index of inflammatory state” means an index which correlates with the inflammatory state, and refers to an index which can be measured or calculated by a blood test, a urine test, or the like. The index of inflammatory state is not particularly limited as long as it is an index which correlates with the inflammatory state. Examples of the index of inflammatory state include substances which are contained in biological samples such as blood and urine obtained from the subject and correlate with the inflammatory state, or concentrations thereof in the biological samples, and more specific examples thereof include C-reactive protein (CRP), inflammatory cytokines, TNF-α, IFNγ, interleukins (IL-1, IL-6, IL-8, IL-12, IL-18, etc.), chemokines, MCP-1 (CCL2), MIP-1α, MCP-2, HMGB-1, α1-globulin fraction, α1-antitrypsin, α1-antichymotrypsin, α1-acid glycoprotein, serum amyloid A (SAA), α2-globulin fraction, haptoglobin, ceruloplasmin, ferritin, number of leukocytes, neutrophil/lymphocyte ratio (NLR), and platelet/lymphocyte ratio (PLR). Also, the index which correlates with the inflammatory state may be, for example, a substance having a function of suppressing an inflammatory symptom, an inflammatory cytokine antagonist, or concentrations thereof in the biological sample. Examples of the index include an anti-inflammatory cytokine (TGF-β), interleukins (IL-4, IL-10, IL-11, etc.), sTNF-R (Soluble Tumor Necrosis Factor-Receptor), and IL-1ra (Interleukin 1 Receptor Antagonist). Further, as the index of inflammatory state, any one of the indexes which correlate with the inflammatory state may be used, or a plurality of the indexes which correlate with the inflammatory state may be used in combination. For example, not only the concentration of CRP in the blood but also the concentration of an interleukin in the blood or urine and the like can be combined as the index of inflammatory state.

In the prognosis prediction method of the present invention, first, (A) the step of measuring an index of nutritional state, sugar metabolism state or inflammatory state of a test subject can be carried out. The index of nutritional state, sugar metabolism state or inflammatory state can be measured by a known method. For example, when the concentration, in a biological sample, of a substance which correlates with the nutritional state, sugar metabolism state or inflammatory state is used as the index of nutritional state, sugar metabolism state or inflammatory state, a biological sample or the like collected from the subject in a blood test or a urine test may be subjected to measurement and calculation of the concentration by a known method. Further, the index of nutritional state may be measured and calculated by a known method such as physical measurement, physical fitness measurement, or motor function measurement. Further, the index of sugar metabolism state may be measured and calculated according to a known procedure such as a sugar tolerance test. For example, when the concentrations of albumin, glucose, C-reactive protein and the like in the blood are used as the index of nutritional state, sugar metabolism state or inflammatory state, the concentrations are examined by a blood test in a periodical health examination or the like, and can be measured according to a well-known procedure.

In the prognosis prediction method of the present invention, the step of determining prognosis for the subject (including prognosis therefor in a ketogenic diet therapy) based on the value of the index of nutritional state, sugar metabolism state or inflammatory state of the subject measured in the above step (A) can be further carried out. In this step, it is indicated that the subject has a poor (or good) prognosis, by comparing the value of the index of nutritional state, sugar metabolism state or inflammatory state of the subject measured in the above step (A) with a reference value regarding the nutritional state, sugar metabolism state or inflammatory state. As used herein, the “comparing” the value of the index of nutritional state, sugar metabolism state or inflammatory state of the subject measured in the above step (A) with the reference value regarding the nutritional state, sugar metabolism state or inflammatory state includes, in addition to cases where an absolute value of the index measured in the above step (A) is compared with an absolute value of the reference value, cases where comparison is made using values obtained by applying an operation such as addition, subtraction, multiplication or division, log conversion, or asin conversion to the value of the index measured in the above step (A) and the reference value, respectively, and cases where an amount or rate of change between the value of the index measured in the above step (A) and the reference value is used. The prognosis prediction method of the present invention may further comprise (B) the step of comparing the value of the index of nutritional state, sugar metabolism state or inflammatory state of the subject with a reference value regarding the nutritional state, sugar metabolism state or inflammatory state and determining that the subject has a poor (or good) prognosis. Further, in the prognosis prediction method of the present invention, the poor prognosis means that the survival rate within a predetermined period is lower, that the cancer progresses (for example, the tumor size increases, and a new lesion appears), and that side effects of the anticancer therapy are caused or increased. In the prognosis prediction method of the present invention, the good prognosis means that the survival rate within a predetermined period is higher, that the cancer regresses (for example, the tumor size decreases, and no new lesion appears), and that side effects of the anticancer therapy are not caused or are reduced.

The reference value regarding the nutritional state can be, for example, a value regarding the nutritional state of the same subject at a certain reference time point, or a value regarding the nutritional state described in any of various guidelines and various reports. For example, it is possible to measure the index of nutritional state before the subject starts an anticancer therapy, to use the value of the index as a reference value, then to measure the index of nutritional state after a lapse of a predetermined period from the start of the anticancer therapy by the subject (carry out the above step (A)), and to compare the measured index with the reference value (carry out the above step (B)). It is also possible to measure the index of nutritional state, at a reference time point, in the subject who has started the anticancer therapy, to use the value of the index as a reference value, then to measure the index of nutritional state after a lapse of a predetermined period (carry out the above step (A)), and to compare the measured index with the reference value (carry out the above step (B)). Further, it is possible to use the value of the index of nutritional state described in any of various guidelines or various as a reference value, then to measure the index of nutritional state for the subject who has started the anticancer therapy (carry out the above step (A)), and to compare the measured index with the reference value (carry out the above step (B)).

The reference value regarding the sugar metabolism state can be, for example, a value regarding the sugar metabolism state of the same subject at a certain reference time point, or a value regarding the sugar metabolism state described in any of various guidelines and various reports. For example, it is possible to measure the index of sugar metabolism state before the subject starts an anticancer therapy, to use the value of the index as a reference value, then to measure the index of sugar metabolism state after a lapse of a predetermined period from the start of the anticancer therapy by the subject (carry out the above step (A)), and to compare the measured index with the reference value (carry out the above step (B)). It is also possible to measure the index of sugar metabolism state at a certain reference time point in the subject who has started the anticancer therapy, to use the value of the index as a reference value, then to measure the index of sugar metabolism state after a lapse of a predetermined period (carry out the above step (A)), and to compare the measured index with the reference value (carry out the above step (B)). Further, it is possible to use the value of the index of sugar metabolism state described in any of various guidelines or various reports as a reference value, then to measure the index of sugar metabolism state for the subject who has started the anticancer therapy (carry out the above step (A)), and to compare the measured index with the reference value (carry out the above step (B)).

The reference value regarding the inflammatory state can be, for example, a value regarding the inflammatory state of the same subject at a certain reference time point, or a value regarding the inflammatory state described in any of various guidelines and various reports. For example, it is possible to measure the index of inflammatory state before the subject starts an anticancer therapy, to use the value of the index as a reference value, then to measure the index of inflammatory state after a lapse of a predetermined period from the start of the anticancer therapy by the subject (carry out the above step (A)), and to compare the measured index with the reference value (carry out the above step (B)). It is also possible to measure the index of inflammatory state, at a reference time point, in the subject who has started the anticancer therapy (carry out the above step (A)), and to compare the measured index with the reference value (carry out the above step (B)). Further, it is possible to use the value of the index of inflammatory state described in any of various guidelines or various reports as a reference value, then to measure the index of inflammatory state of the subject who has started the anticancer therapy (carry out the above step (A)), and to compare the measured index with the reference value (carry out the above step (B)).

For example, in the case where the index of nutritional state measured in the above step (A) is the concentration of albumin in the blood, it is indicated that the subject has a poor prognosis when the concentration of albumin in the blood is lower than the reference value in the subject, and that the subject has a good prognosis when the concentration of albumin in the blood exceeds the reference value in the subject. For example, in the case where the index of sugar metabolism state measured in the above step (A) is the concentration of glucose in the blood, it is indicated that the subject has a poor prognosis when the blood sugar level is higher than the reference value in the subject, and that the subject has a good prognosis when the blood sugar level is lower than the reference value in the subject. For example, in the case where the index of inflammatory state measured in the above step (A) is the concentration of C-reactive protein (CRP) in the blood, it is indicated that the subject has a poor prognosis when the concentration of CRP in the blood is higher than the reference value in the subject, and that the subject has a good prognosis when the concentration of CRP in the blood is lower than the reference value in the subject.

In the prognosis prediction method of the present invention, the index used for predicting prognosis may be any one of the index of nutritional state, the index of sugar metabolism state, and the index of inflammatory state, or a combination of some or all of the indexes. When step (B) is carried out using a combination of two indexes, it may be determined that the subject has a poor (or good) prognosis, by defining the reference value for each index and comparing any one of the indexes with the corresponding reference value. Also, when step (B) is carried out using a combination of three indexes, it may be determined that the subject has a poor (or good) prognosis, by defining the reference value for each index and comparing two or three (preferably, at least one) of the indexes with the corresponding reference values. Further, in the prognosis prediction method of the present invention, in order to improve the accuracy of prognosis prediction, any one or more of the index of nutritional state, the index of sugar metabolism state, and the index of inflammatory state may be subjected to weighting by a coefficient and then used in the prognosis prediction. For example, in order to improve the accuracy of prognosis prediction, any one or more of the index of nutritional state, the index of sugar metabolism state, and the index of inflammatory state may be subjected to a combination of weighting by a coefficient and an operation such as addition, subtraction, multiplication or division, log conversion, or asin conversion, and then used in the prognosis prediction. The operation may also be performed artificially, and functions of AI (artificial intelligence) and the like may also be utilized.

The above steps (A) and (B) can be carried out on a cancer patient at any timing. For example, when the value of the index of nutritional state, sugar metabolism state or inflammatory state described in any of various guidelines and various reports is used as the reference value, the above steps (A) and (B) can be carried out at any timing, for the index of nutritional state, sugar metabolism state or inflammatory state to be compared with the reference value. Also, for example, when the index of nutritional state, sugar metabolism state or inflammatory state at a certain reference time point is measured, and the value of the index is used as the reference value, the above steps (A) and (B) can be carried out after a lapse of a predetermined period from the reference time point (for example, after a lapse of any period between 1 week and 24 months).

For the cancer patient on whom the anticancer therapy has been performed, for example, the value of the index of nutritional state, sugar metabolism state or inflammatory state before start of the anticancer therapy may be used as the reference value, and the above steps (A) and (B) may be carried out after a lapse of a predetermined time from the start of the anticancer therapy (for example, after a lapse of any period between 1 week and 24 months). Or, the index of nutritional state, sugar metabolism state or inflammatory state at a certain reference time point after start of the anticancer therapy may be measured, the value of the index may be used as the reference value, and the above steps (A) and (B) may be carried out after a lapse of a predetermined time from the reference time point (for example, after a lapse of any period between 1 week and 24 months). Also, the value of the index of nutritional state, sugar metabolism state or inflammatory state described in any of various guidelines and various reports can be used as the reference value, and the above steps (A) and (B) can be carried out at any timing. The predetermined period described above can be defined so as to correspond to the period when the reference value is defined, in order to improve the accuracy of prognosis prediction.

According to the prognosis prediction method of the present invention, the prognosis for the cancer patient can be predicted or determined. Thus, the prognosis prediction method of the present invention is useful in providing appropriate information for making decision when determining the treatment policy of the anticancer therapy. Specifically, the prognosis prediction method of the present invention can be used accessorily in the treatment of cancer by the anticancer therapy, and whether the prognosis for the subject is poor or not (or the prognosis for the subject is good or not) may be finally decided, by a doctor, in combination with any other finding in some cases. According to the prognosis prediction method of the present invention, the prognosis for the cancer patient in the ketogenic diet therapy can be predicted or determined. Thus, the prognosis prediction method of the present invention is useful in providing appropriate information for making decision when determining the treatment policy of the ketogenic diet therapy. That is, the prognosis prediction method of the present invention can be used accessorily in the treatment of cancer by the ketogenic diet therapy.

### <<Method for Predicting Effectiveness>>

In the method for predicting effectiveness of an anticancer therapy of the present invention, at least one index selected from the group consisting of an index of nutritional state, an index of sugar metabolism state, and an index of inflammatory state is used as an index of effectiveness of an anticancer therapy.

In the method for predicting effectiveness of an anticancer therapy of the present invention, first, (C) the step of measuring an index of nutritional state, sugar metabolism state or inflammatory state of a test subject can be carried out. The index of nutritional state, sugar metabolism state or inflammatory state and measurement thereof are as described for the prognosis prediction method of the present invention.

In the method for predicting effectiveness of an anticancer therapy of the present invention, the step of determining effectiveness of an anticancer therapy (including a ketogenic diet therapy) for the subject based on the value of the index of nutritional state, sugar metabolism state or inflammatory state of the subject measured in the above step (C) can be further carried out. In this step, it is indicated that the effectiveness of the anticancer therapy is low (or high) for the subject, by comparing the value of the index of nutritional state, sugar metabolism state or inflammatory state of the subject measured in the above step (C) with a reference value regarding the nutritional state, sugar metabolism state or inflammatory state. As used herein, the “comparing” the value of the index of nutritional state, sugar metabolism state or inflammatory state of the subject measured in the above step (C) with the reference value regarding the nutritional state, sugar metabolism state or inflammatory state includes, in addition to cases where an absolute value of the index measured in the above step (C) is compared with an absolute value of the reference value, cases where comparison is made using values obtained by applying an operation such as addition, subtraction, multiplication or division, log conversion, or asin conversion to the value of the index measured in the above step (C) and the reference value, respectively, and cases where an amount or rate of change between the value of the index measured in the above step (C) and the reference value is used. The method for predicting effectiveness of an anticancer therapy of the present invention may further comprise (D) the step of comparing the value of the index of nutritional state, sugar metabolism state or inflammatory state of the subject with a reference value regarding the nutritional state, sugar metabolism state or inflammatory state and determining that the effectiveness of the anticancer therapy is low (or high) for the subject. Further, in the method for predicting effectiveness of an anticancer therapy of the present invention, the low effectiveness of the anticancer therapy means that the cancer recovery rate within a predetermined period is lower, that the cancer progresses (for example, the tumor size increases, and a new lesion appears), and that side effects of the anticancer therapy are caused or increased. In the method for predicting effectiveness of an anticancer therapy of the present invention, the high effectiveness of the anticancer therapy means that the cancer recovery rate within a predetermined period is higher, that the cancer regresses (for example, the tumor size decreases, and no new lesion appears), and that side effects of the anticancer therapy are not caused or are reduced.

The reference value regarding the nutritional state, the reference value regarding the sugar metabolism state, or the reference value regarding the inflammatory state in the method for predicting effectiveness of an anticancer therapy of the present invention is as described for the prognosis prediction method of the present invention. For example, in the case where the index of nutritional state measured in the above step (C) is the concentration of albumin in the blood, it is indicated that the effectiveness of the anticancer therapy is low when the concentration of albumin in the blood is lower than the reference value in the subject, and that the effectiveness of the anticancer therapy is high when the concentration of albumin in the blood is higher than the reference value in the subject. For example, in the case where the index of sugar metabolism state measured in the above step (C) is the concentration of glucose in the blood, it is indicated that the effectiveness of the anticancer therapy is low when the blood sugar level is higher than the reference value in the subject, and that the effectiveness of the anticancer therapy is high when the blood sugar level is lower than the reference value in the subject. For example, in the case where the index of inflammatory state measured in the above step (C) is the concentration of C-reactive protein (CRP) in the blood, it is indicated that the effectiveness of the anticancer therapy is low when the concentration of CRP in the blood is higher than the reference value in the subject, and that the effectiveness of the anticancer therapy is high when the concentration of CRP in the blood is lower than the reference value in the subject.

In the method for predicting effectiveness of an anticancer therapy of the present invention, the index used for predicting effectiveness may be any one of the index of nutritional state, the index of sugar metabolism state, and the index of inflammatory state, or a combination of some or all of the indexes. When step (D) is carried out using a combination of two indexes, it may be determined that the effectiveness of the anticancer therapy is low (or high) for the subject, by defining the reference value for each index and comparing any one of the indexes with the corresponding reference value. Also, when step (D) is carried out using a combination of three indexes, it may be determined that the effectiveness of the anticancer therapy is low (or high) for the subject, by defining the reference value for each index and comparing two or three (preferably, at least one) indexes with the corresponding reference values. Further, in the method for predicting effectiveness of an anticancer therapy of the present invention, any one or more of the index of nutritional state, the index of sugar metabolism state, and the index of inflammatory state may be subjected to weighting by a coefficient and then used in the effectiveness prediction, in order to improve the accuracy of effectiveness prediction. For example, any one or more of the index of nutritional state, the index of sugar metabolism state, and the index of inflammatory state may be subjected to a combination of weighting by a coefficient and an operation such as addition, subtraction, multiplication or division, log conversion, or asin conversion, and then used in the effectiveness prediction, in order to improve the accuracy of effectiveness prediction. The operation may also be performed artificially, and functions of AI (artificial intelligence) and the like may also be utilized.

The above steps (C) and (D) can be carried out on a cancer patient at any timing. For example, when the value of the index of nutritional state, sugar metabolism state or inflammatory state described in any of various guidelines and various reports is used as the reference value, the above steps (C) and (D) can be carried out at any timing, for the index of nutritional state, sugar metabolism state or inflammatory state to be compared with the reference value. Also, for example, when the index of nutritional state, sugar metabolism state or inflammatory state at a certain reference time point is measured, and the value of the index is used as the reference value, the above steps (C) and (D) can be carried out after a lapse of a predetermined period from the reference time point (for example, after a lapse of any period between 1 week and 24 months).

For the cancer patient on whom the anticancer therapy has been performed, the value of the index of nutritional state, sugar metabolism state or inflammatory state before start of the anticancer therapy may be used as the reference value, and the above steps (C) and (D) may be carried out after a lapse of a predetermined time from the start of the anticancer therapy (for example, after a lapse of any period between 1 week and 24 months), or the index of nutritional state, sugar metabolism state or inflammatory state at a certain reference time point after start of the anticancer therapy may be measured, the value of the index may be used as the reference value, and the above steps (C) and (D) may be carried out after a lapse of a predetermined time from the reference time point (for example, after a lapse of any period between 1 week and 24 months). Also, the value of the index of nutritional state, sugar metabolism state or inflammatory state described in any of various guidelines and various reports can be used as the reference value, and the above steps (C) and (D) can be carried out at any timing. The predetermined period described above can be defined so as to correspond to the period when the reference value is defined, in order to improve the accuracy of prediction of the effectiveness of the anticancer therapy.

According to the method for predicting effectiveness of an anticancer therapy of the present invention, the effectiveness of the anticancer therapy in the cancer patient can be predicted or determined. Thus, the method for predicting effectiveness of an anticancer therapy of the present invention is useful in providing appropriate information for making decision when determining the treatment policy of the anticancer therapy. Specifically, the method for predicting effectiveness of an anticancer therapy of the present invention can be used accessorily in the treatment of cancer by the anticancer therapy, and whether or not the anticancer therapy is effective for the subject may be finally decided, by a doctor, in combination with any other finding in some cases. Also, according to the method for predicting effectiveness of an anticancer therapy of the present invention, the effectiveness of the ketogenic diet therapy in the cancer patient can be predicted or determined. Thus, the method for predicting effectiveness of an anticancer therapy of the present invention is useful in providing appropriate information for making decision when determining the treatment policy of the ketogenic diet therapy. That is, the method for predicting effectiveness of an anticancer therapy of the present invention can be used accessorily in the treatment of cancer by the ketogenic diet therapy.

### <<Method for Selecting Appropriate Therapy>>

In the selection method of the present invention, at least one index selected from the group consisting of an index of nutritional state, an index of sugar metabolism state, and an index of inflammatory state is used as an index for selection of an appropriate therapy for the cancer patient.

In the selection method of the present invention, first, (E) the step of measuring an index of nutritional state, sugar metabolism state or inflammatory state of a test subject can be carried out. The index of nutritional state, sugar metabolism state or inflammatory state and measurement thereof are as described for the prognosis prediction method of the present invention.

In the selection method of the present invention, the step of determining effectiveness of an anticancer therapy (including a ketogenic diet therapy) for the subject based on the value of the index of nutritional state, sugar metabolism state or inflammatory state of the subject measured in the above step (E) can be further carried out. In this step, it is indicated that the effectiveness of the anticancer therapy is low (or high) for the subject, by comparing the value of the index of nutritional state, sugar metabolism state or inflammatory state of the subject measured in the above step (E) with a reference value regarding the nutritional state, sugar metabolism state or inflammatory state. In the selection method of the present invention, the low effectiveness of the anticancer therapy means that the cancer recovery rate within a predetermined period is lower, that the cancer progresses (for example, the tumor size increases, and a new lesion appears), and that side effects of the anticancer therapy are caused or increased. In the selection method of the present invention, the high effectiveness of the anticancer therapy means that the cancer recovery rate within a predetermined period is higher, that the cancer regresses (for example, the tumor size decreases, and no new lesion appears), and that side effects of the anticancer therapy are not caused or are reduced. Thus, when the effectiveness of the anticancer therapy in the subject is low, any of anticancer therapies other than the anticancer therapy in the subject (e.g., surgical therapies such as resection/extraction, chemotherapies, radiation therapies, cancer immunotherapies such as CAR-T cell therapies, and diet therapies such as ketone diet therapies, and combinations of some or all of them) can be selected as an appropriate therapy. In addition, when the effectiveness of the anticancer therapy in the subject is high, the anticancer therapy in the subject can be continued or discontinued. As used herein, the “comparing” the value of the index of nutritional state, sugar metabolism state or inflammatory state of the subject measured in the above step (E) with the reference value regarding the nutritional state, sugar metabolism state or inflammatory state includes, in addition to cases where an absolute value of the index measured in the above step (E) is compared with an absolute value of the reference value, cases where comparison is made using values obtained by applying an operation such as addition, subtraction, multiplication or division, log conversion, or asin conversion to the value of the index measured in the above step (E) and the reference value, respectively, and cases where an amount or rate of change between the value of the index measured in the above step (E) and the reference value is used. The selection method of the present invention may further comprise (F) the step of comparing the value of the index of nutritional state, sugar metabolism state or inflammatory state of the subject with a reference value regarding the nutritional state, sugar metabolism state or inflammatory state and determining that it is desired to select an anticancer therapy other than the anticancer therapy in the subject (or it is desired to continue or discontinue the anticancer therapy in the subject).

The reference value regarding the nutritional state, the reference value regarding the sugar metabolism state, and the reference value regarding the inflammatory state in the selection method of the present invention are as described for the prognosis prediction method of the present invention. For example, in the case where the index of nutritional state measured in the above step (E) is the concentration of albumin in the blood, it is indicated that it is desired to select an anticancer therapy other than the anticancer therapy in the subject when the concentration of albumin in the blood is lower than the reference value in the subject, and that it is desired to continue or discontinue the anticancer therapy in the subject when the concentration of albumin in the blood is higher than the reference value in the subject. For example, in the case where the index of sugar metabolism state measured in the above step (E) is the concentration of glucose in the blood, it is indicated that it is desired to select an anticancer therapy other than the anticancer therapy in the subject when the blood sugar level is higher than the reference value in the subject, and that it is desired to continue or discontinue the anticancer therapy in the subject when the blood sugar level is lower than the reference value in the subject. For example, in the case where the index of inflammatory state measured in the above step (E) is the concentration of C-reactive protein (CRP) in the blood, it is indicated that it is desired to select an anticancer therapy other than the anticancer therapy in the subject when the concentration of CRP in the blood is higher than the reference value in the subject, and that it is desired to continue or discontinue the anticancer therapy in the subject when the concentration of CRP in the blood is lower than the reference value in the subject.

In the selection method of the present invention, the index used for selection may be any one of the index of nutritional state, the index of sugar metabolism state, and the index of inflammatory state, or a combination of some or all of the indexes. When step (F) is carried out using a combination of two indexes, it may be determined that it is desired to select an anticancer therapy other than the anticancer therapy in the subject (or it is desired to continue or discontinue the anticancer therapy in the subject), by defining the reference value for each index and comparing any one of the indexes with the corresponding reference value. Also, when step (F) is carried out using a combination of three indexes, it may be determined that it is desired to select an anticancer therapy other than the anticancer therapy in the subject (or it is desired to continue or discontinue the anticancer therapy in the subject), by defining the reference value for each index and comparing two or three (preferably, at least one) indexes with the corresponding reference values. Further, in the selection method of the present invention, any one or more of the index of nutritional state, the index of sugar metabolism state, and the index of inflammatory state may be subjected to weighting by a coefficient and then used in the selection of an appropriate therapy for the cancer patient, in order to improve the accuracy of selection. For example, in order to more appropriately select an appropriate therapy for the cancer patient, any one or more of the index of nutritional state, the index of sugar metabolism state, and the index of inflammatory state may be subjected to a combination of weighting by a coefficient and an operation such as addition, subtraction, multiplication or division, log conversion, or asin conversion, and then used in the selection of the appropriate therapy for the cancer patient. The operation may also be performed artificially, and functions of AI (artificial intelligence) and the like may also be utilized.

The above steps (E) and (F) can be carried out on a cancer patient at any timing. For example, when the value of the index of nutritional state, sugar metabolism state or inflammatory state described in any of various guidelines and various reports is used as the reference value, the above steps (E) and (F) can be carried out at any timing, for the index of nutritional state, sugar metabolism state or inflammatory state to be compared with the reference value. Also, for example, when the index of nutritional state, sugar metabolism state or inflammatory state at a certain reference time point is measured, and the value of the index is used as the reference value, the above steps (E) and (F) can be carried out after a lapse of a predetermined period from the reference time point (for example, after a lapse of any period between 1 week and 24 months).

For the cancer patient on whom the anticancer therapy has been performed, the value of the index of nutritional state, sugar metabolism state or inflammatory state before start of the anticancer therapy may be used as the reference value, and the above steps (E) and (F) may be carried out after a lapse of a predetermined time from the start of the anticancer therapy (for example, after a lapse of any period between 1 week and 24 months), or the index of nutritional state, sugar metabolism state or inflammatory state at a certain reference time point after start of the anticancer therapy may be measured, the value of the index may be used as the reference value, and the above steps (E) and (F) may be carried out after a lapse of a predetermined time from the reference time point (for example, after a lapse of any period between 1 week and 24 months). Also, the value of the index of nutritional state, sugar metabolism state or inflammatory state described in any of various guidelines and various reports can be used as the reference value, and the above steps (E) and (F) can be carried out at any timing. The predetermined period described above can be defined so as to correspond to the period when the reference value is defined, in order to improve the accuracy of selection of an appropriate therapy for the cancer patient.

According to the selection method of the present invention, it is possible to select an appropriate therapy for a cancer patient. Thus, the selection method of the present invention is useful in providing appropriate information for making decision when determining the treatment policy of the anticancer therapy. Specifically, the selection method of the present invention can be used accessorily in the treatment of cancer by the anticancer therapy, and whether or not any other anticancer therapy than the anticancer therapy in the subject should be selected may be finally decided, by a doctor, in combination with any other finding in some cases. Also, according to the selection method of the present invention, it is possible to select an appropriate therapy for a cancer patient in a ketogenic diet therapy. Thus, the selection method of the present invention is useful in providing appropriate information for making decision when determining the treatment policy of the ketogenic diet therapy. Specifically, the selection method of the present invention can be used accessorily in the treatment of cancer by the ketogenic diet therapy, and whether or not any other anticancer therapy than the ketogenic diet therapy should be selected for the subject may be finally decided, by a doctor, in combination with any other finding in some cases.

According to another aspect of the present invention, there is provided a method for treating a cancer patient, wherein the method for selecting of the present invention is carried out to select an appropriate anticancer therapy for the cancer patient, and the selected anticancer therapy is performed on the patient. According to still another aspect of the present invention, there is provided a method for treating a cancer patient in a ketogenic diet therapy, wherein the method for selecting of the present invention is carried out to select an appropriate anticancer therapy for the cancer patient in the ketogenic diet therapy, and the selected anticancer therapy is performed on the patient. The selection of an appropriate anticancer therapy in the treatment method of the present invention can be performed according to the method for selecting of the present invention as described above.

According to the present invention, at least one of the index of nutritional state, the index of sugar metabolism state, and the index of inflammatory state can be used as an index to predict the prognosis for a cancer patient, to predict the effect of an anticancer therapy in the cancer patient, and further to select an appropriate therapy for the cancer patient. Therefore, the present invention is advantageous in that the anticancer therapy can be performed more efficiently, which in turn leads to improvement of patient QOL and provision of a therapy tailored to an individual patient (personalized nutrition, precision medicine, etc.). The present invention is advantageous also in that at least one of the index of nutritional state, the index of sugar metabolism state, and the index of inflammatory state which can be measured by a blood test, a urine test, physical measurement, physical fitness measurement, motor function measurement, or the like can be used as an index to predict the prognosis for a cancer patient, to predict the effect of an anticancer therapy in the cancer patient, and further to select an appropriate therapy for the cancer patient.

Also, according to the present invention, at least one of the index of nutritional state, the index of sugar metabolism state, and the index of inflammatory state in a terminal cancer patient on whom a saccharide restricted high fat diet therapy has been performed can be used as an index to predict the prognosis for a cancer patient, to predict the effect of a ketogenic diet therapy in the cancer patient, and further to select an appropriate therapy for the cancer patient. Therefore, the present invention is advantageous in that the anticancer therapy in the ketogenic diet therapy can be performed more efficiently, which in turn leads to improvement of patient QOL and provision of a therapy tailored to an individual patient (personalized nutrition, precision medicine, etc.). The present invention is advantageous also in that at least one of the index of nutritional state, the index of sugar metabolism state, and the index of inflammatory state which can be measured by a blood test, a urine test, physical measurement, physical fitness measurement, motor function measurement, or the like can be used as an index to predict the prognosis for a cancer patient in a ketogenic diet therapy, to predict the effect of the ketogenic diet therapy in the cancer patient, and further to select an appropriate therapy for the cancer patient in the ketogenic diet therapy.

### EXAMPLES

Hereinafter, the present invention will be described in more detail by way of the following examples, but is not limited thereto.

### Example 1: Effect of Predicting Prognosis of Cancer Using Combination of Concentration of Albumin in Blood, Blood Sugar Level and Concentration of CRP in Blood as Index

### (1) Selection of Subjects

Subjects were 55 (24 males and 31 females) stage 4 cancer patients with a performance status (PS) of 0 to 2, who were capable of oral intake. The subjects had an average age of 55.8±12.1 years, an average height of 162.3±8.7 cm, and an average body weight of 54.7±12.1 kg. The diseases were lung cancer in 12 cases, colorectal cancer in 9 cases, breast cancer in 5 cases, bladder cancer in 2 cases, ovarian cancer in 2 cases, and other cancers in 21 cases. The treatment histories were chemotherapy in 42 cases, surgery in 32 cases, and radiation in 17 cases.

### (2) Test Method

In addition to normal treatment (a surgical therapy, a chemotherapy, a radiation therapy or the like), a ketogenic diet therapy was performed on patients with various cancers as listed in the above item (1). Explanation was given that temporary hypoglycemia, nausea, fatigue, or the like would appear at the time of introduction of the ketogenic diet. Actual nutritional guidance was given under the guidance of a nutritionist who had given the guidance of a ketogenic diet to epilepsy patients for a long time. Persons who would cook the ketogenic diet also attended the nutritional guidance. The details of the ketogenic diet therapy were as follows.

A. For the first week, the goal was: a calorie of about 30 kcal/kg body weight, no restriction on lipid or protein, and a daily intake amount of carbohydrate (carbohydrate other than dietary fibers, corresponding to saccharide; the same applies hereinafter) of about 10 g or less, based on the real body weight. Specifically, the goal was: a daily caloric intake of about 1500 kcal and a ratio of 140 g of lipid:60 g of protein:10 g of saccharide, based on a real body weight of 50 kg, and a ketogenic ratio [lipid (g):(protein (g)+saccharide (g))] of 2:1. Other nutrients could be fed without restriction. Necessary trace elements and/or vitamins were appropriately fed through the use of supplements or the like. At the time of introduction of the ketogenic diet, meals in accordance with the menu prepared by a nutritionist were fed.

B. For the second week to the third month, the contents of the meals were adjusted with reference to the blood ketone body values. Concerning the blood ketone body values, guidance was given so that acetoacetic acid was not 500 μmol/L, or more and β-hydroxybutyric acid was not 1000 μmol/L or less, and, if possible, the target values were set to 1000 μmol/L or more for acetoacetic acid and 2000 μmol/L or more for β-hydroxybutyric acid. The goal was: a daily intake amount of carbohydrate of 20 g or less, a daily caloric intake of 1400 to 1600 kcal, a ratio of 120 to 140 g of lipid:70 g of protein:20 g of carbohydrate, and a ketogenic ratio [lipid (g):(protein (g)+saccharide (g))] of 2:1 to 1:1. At the time of calorie supply, “Ketonformula” (manufactured by Meiji Co., Ltd.) or “MCT Oil” (manufactured by The Nisshin OilliO Group, Ltd.) was used.

C. After the third month, the daily intake amount of carbohydrate was set to 30 g or less when the single intake amount thereof was 10 g/intake, and others were in accordance with (2).

The ketogenic diet (containing 75 to 80% of lipid) has been administered to child epileptic patients for a long term, so that the safety thereof has been confirmed. Thus, the diet is described in the 2010 edition of the COCHRANE LIBRARY. The document was also referred to as a reference for the actual policy. It has been confirmed that that temporal nausea, fatigue, hypoglycemia or the like, which may be observed at the early stage of introduction, can be sufficiently dealt with. Since this is a high lipid diet, there is a possibility that a certain percentage of patients cannot continue the ketogenic diet due to their preference. It is possible to deal with such issues in cooperation with the nutritionist.

### (3) Evaluation Method

As a primary endpoint, the therapeutic effect of the ketogenic diet therapy on cancer was evaluated using PET-CT images before and after the introduction of the ketogenic diet Specifically, the therapeutic effect was determined by comparing images before the start of the ketogenic diet and 3 months after the start of the ketogenic diet (hereinafter sometimes referred to simply as “3 months after the start of the test”), in accordance with the RECIST guideline (Therasse P, et al., J Natl Cancer Inst, 2000, Vol 92, No. 3, 205-216). The therapeutic effects obtained were classified into: “complete remission (CR)” in which tumor completely disappeared; “partial response (PR)” in which tumor was smaller by 30% or more; “stable disease” in which the tumor size was unchanged; and “progressive disease (PD)” in which the sum of tumor diameters increased by 20% or more and the sum of tumor diameters increased by 5 mm or more in absolute value, or a new lesion appeared. In addition, as a secondary endpoint, the survival rate for about 6 years after the start of the test was evaluated using the survival rate curve by the Kaplan-Meier method.

Three (3) months after the start of the test, blood was collected from the subjects to measure the concentration of albumin in the blood, the blood sugar level, and the concentration of CRP in the blood. The concentration of albumin in the blood was scored: 0 points for 4.0 g/dl or more and 1 point for less than 4.0 g/dl. The blood sugar level was scored: 0 points for 90 mg/dl or less and 1 point for more than 90 mg/dl. The concentration of CRP in the blood was scored: 0 points for 0.5 mg/dl or less and 1 point for more than 0.5 mg/dl. The total score was calculated for each subject, and the subjects were divided into groups in which the total score of the combination of the concentration of albumin in the blood, the blood sugar level, and the concentration of CRP in the blood (hereinafter, sometimes referred to simply as “combination score”) was 0, 1, 2 or 3 points. In addition, the glucose-ketogenic index 3 months after the start of the test was calculated according to the literature (Meidenbauer et al., Nutr Metab, 2015 Mar. 11; 12:12). The transition of the gastrointestinal symptom score was evaluated using the Gastrointestinal Symptom Rating Scale (hereinafter, sometimes referred to simply as “GSRS score”). In addition, the transition of the general condition scale of QOL (QOL score for cancer treatment) was evaluated using a questionnaire form (EORTC QLQ-C30).

### (4) Results

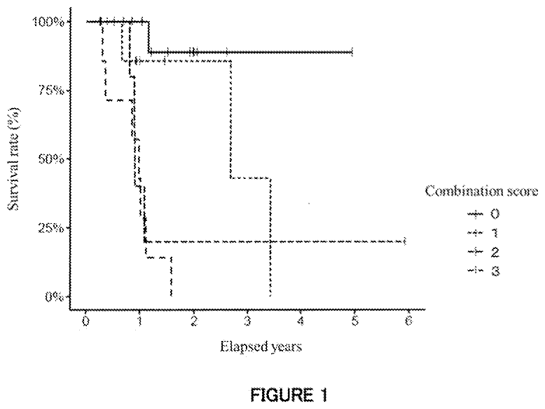

Of the 55 subjects, 5 cases were subjects on whom the test was not performed, 11 cases were subjects on whom the test was discontinued, and 2 cases were subjects who were excluded from the analysis. The effect of continuing the ketogenic diet for 3 months was evaluated for 37 cases excluding these cases. The glucose-ketogenic index 3 months after the start of the test achieved moderate or higher ketosis in 31 cases, but no significant change was found in the GSRS score or the general condition scale of QOL. The cancer treatment effects were: complete remission (CR) in 0 cases, partial response (PR) in 4 cases, stable disease (SD) in 20 cases, progressive disease (PD) in 12 cases, and NP (Not performed) in 1 case. The survival rate was as shown in FIG. 1, and the number at risk (the number of patients who were alive at that time) was as indicated in Table 1.

TABLE 1
Number at risk (number of patients who were alive
at that time) in combination score groups

| | Years lapsed after start of test | | | | | | |
|---|---|---|---|---|---|---|---|
| | 0 years | 1 year | 2 years | 3 years | 4 years | 5 years | 6 years |
| 0-points | 14 cases | 10 cases | 3 cases | 1 case | 1 case | 0 cases | 0 cases |
| group | | | | | | | |
| 1-point | 8 cases | 4 cases | 2 cases | 1 case | 0 cases | 0 cases | 0 cases |
| group | | | | | | | |
| 2-points | 7 cases | 2 cases | 1 case | 1 case | 1 case | 1 case | 0 cases |
| group | | | | | | | |
| 3-points | 8 cases | 3 cases | 0 cases | 0 cases | 0 cases | 0 cases | 0 cases |
| group | | | | | | | |

The median survival period was 979 days (maximally, 2164 days), and 22 subjects were alive at the end of the test. In grouping based on the scores of the concentration of albumin in the blood, the blood sugar level, and the concentration of CRP in the blood 3 months after the start of the test, the total score was 0 points in 14 cases, 1 point in 8 cases, 2 points in 7 cases and 3 points in 8 cases. It was confirmed, from FIG. 1, that a significant difference in survival period was observed depending on the score (log rank test, p<0.001). It was also confirmed that the anticancer therapy was more effective in the group of cancer patients with a lower combination score of the concentration of albumin in the blood, the blood sugar level, and the concentration of CRP in the blood. In addition, since prognosis can be predicted according to the combination score of the concentration of albumin in the blood, the blood sugar level, and the concentration of CRP in the blood, it was confirmed that an appropriate therapy could be selected for cancer patients. From the above results, it was demonstrated that the combination of the concentration of albumin in the blood, the blood sugar level, and the concentration of CRP in the blood of the patients on whom the ketogenic diet therapy had been performed for 3 months could be used as an index to predict prognoses for the cancer patients (especially, prognosis for the cancer patients in the ketogenic diet therapy), to select a therapy in the cancer patients (especially, for the cancer patients in the ketogenic diet therapy), and to predict effectiveness of the anticancer therapy in the cancer patients (especially, effectiveness of the ketogenic diet therapy).

1. A method for predicting prognosis for a cancer patient, wherein at least one index selected from the group consisting of an index of nutritional state, an index of sugar metabolism state, and an index of inflammatory state in the cancer patient is used as an index of prognosis.

2. A method for predicting effectiveness of an anticancer therapy in a cancer patient, wherein at least one index selected from the group consisting of an index of nutritional state, an index of sugar metabolism state, and an index of inflammatory state in the cancer patient is used as an index of effectiveness of an anticancer therapy in the cancer patient.

3. A method for selecting an appropriate therapy for a cancer patient, wherein at least one index selected from the group consisting of an index of nutritional state, an index of sugar metabolism state, and an index of inflammatory state in the cancer patient is used as an index for selection of an appropriate therapy for the cancer patient.

4. The method according to claim 1, wherein a combination of the index of nutritional state, the index of sugar metabolism state, and the index of inflammatory state is used as the index.

5. The method according to claim 1, wherein the index of nutritional state is a concentration of albumin in the blood.

6. The method according to claim 1, wherein the index of sugar metabolism state is a concentration of glucose in the blood.

7. The method according to claim 1, wherein the index of inflammatory state is a concentration of C-reactive protein (CRP) in the blood.

8. The method according to claim 1, wherein the cancer patient is a refractory cancer patient having a performance status of 2 or less.

9. The method according to claim 1, wherein the cancer patient is a patient on whom a ketogenic diet therapy has been performed.

10. The method according to claim 2, wherein a combination of the index of nutritional state, the index of sugar metabolism state, and the index of inflammatory state is used as the index.

11. The method according to claim 2, wherein the index of nutritional state is a concentration of albumin in the blood.

12. The method according to claim 2, wherein the index of sugar metabolism state is a concentration of glucose in the blood.

13. The method according to claim 2, wherein the index of inflammatory state is a concentration of C-reactive protein (CRP) in the blood.

14. The method according to claim 2, wherein the cancer patient is a refractory cancer patient having a performance status of 2 or less.

15. The method according to claim 2, wherein the cancer patient is a patient on whom a ketogenic diet therapy has been performed.

16. The method according to claim 3, wherein a combination of the index of nutritional state, the index of sugar metabolism state, and the index of inflammatory state is used as the index.

17. The method according to claim 3, wherein the index of nutritional state is a concentration of albumin in the blood.

18. The method according to claim 3, wherein the index of sugar metabolism state is a concentration of glucose in the blood.

19. The method according to claim 3, wherein the index of inflammatory state is a concentration of C-reactive protein (CRP) in the blood.

20. The method according to claim 3, wherein the cancer patient is a refractory cancer patient having a performance status of 2 or less.

21. The method according to claim 3, wherein the cancer patient is a patient on whom a ketogenic diet therapy has been performed.

22. A method for treating a cancer patient, which comprises the steps of carrying out the method according to claim 3 to select an appropriate anticancer therapy for the cancer patient, and performing the selected anticancer therapy on the patient.

23. The method according to claim 22, wherein the cancer patient is a patient on whom a ketogenic diet therapy has been performed.
